(19) United States
(12) Patent Application Publication
(10) Pub. No.: US 2009/0028749 A1
(43) Pub. Date: Jan. 29, 2009

(54) Process and device for controlling microbial contamination in dampening agent cycles

(75) Inventor: Helmut Sander, Erftstadt (DE)

Correspondence Address: OHLANDT, GREELEY, RUGGIERO & PERLE, LLP, ONE LANDMARK SQUARE, 10TH FLOOR, STAMFORD, CT, 06901, US

(73) Assignee: FELIX BOETTCHER GMBH & CO., KG.

(21) Appl. No.: 11/918,529

(22) PCT Filed: Apr. 25, 2006
(86) PCT No.: PCT/EP2006/061817

§ 371 Date: Oct. 15, 2007

(30) Foreign Application Priority Data
Apr. 26, 2005 (EP) 05103372.8

(51) Int. Cl. A61L 2/03

(52) U.S. Cl. 422/22; 422/127

(57) ABSTRACT

A process for reducing the microbial contamination of dampening agent cycles, comprising the step of electrochemically treating the dampening agent.

### BACKGROUND

1. Field of the Disclosure

The present disclosure relates to a process and device for controlling and limiting microbial contamination in dampening agent cycles and other aqueous media, especially in the printing field.

2. Discussion of the Background Art

A dampening agent (dampening solution) is employed for keeping hydrophilic regions of printing plates hydrophilic in places where no printing ink is to be received.

The dampening solution is applied to the printing plate directly or indirectly via the dampening unit, whereby the printing ink takes up a limited amount of dampening agent to form an emulsion. The dampening solution contains components which keep the nonprinting parts of the printing plate hydrophilic, whereby the reception of printing ink in this region is prevented.

This method is employed, in particular, in planographic printing methods, in which the printing and nonprinting regions of the printing forme are almost in a plane. These printing methods are based on the dissimilar physico-chemical behavior of particular substances in the coating of the printing plate which are ink-receiving or ink-repelling.

In offset printing, this is a thin printing plate (e.g., of aluminum) on the surface of which there are ink-receiving (lipophilic) image areas and non-image (hydrophilic) areas.

A printing machine consumes on the order of from 10 to 150 liters of dampening solution per hour. The dampening solution is typically used in a slightly cooled condition.

In addition to water, the dampening agent typically contains pH-regulating substances (buffers), preservatives (e.g., biocides), corrosion inhibitors, plate-protection components, and optionally wetting agents. These components are usually contained in a dampening agent concentrate, which is then diluted with water to give a predetermined concentration. The ready-to-use dampening agent is circulated in a closed cycle. The individual dampening units of the printing machine are connected with the dampening agent supply system through a circulation line.

In the supply line, the dampening agent is supplied to the dampening unit. In the return line, the excess dampening agent contaminated with ink and paper particles flows back to the reprocessing plant. Depending on the design of the plant, the returning dampening agent is purified from soil particles through a filter material or filter bags made of synthetic fibers before it flows into the sump of the plant. The cooling device by means of which the temperature of the dampening agent is kept constant at a predetermined value, which is typically within a range of from 9 to 15° C., is also usually attached to this dampening agent reprocessing equipment.

During the printing process, dampening agent is constantly consumed and replenished by metering fresh dampening agent. Despite of cooling and filtering, every now and then, the germ load in the dampening agent cycle is highly increased. The soil particles from the printing process, especially cellulose from the paper coating, but also the buffers employed, provide favorable growth conditions for the germs.

Bioflims are formed in the pipelines, filters, dampening units and in the sump of the dampening agent supply plant. As the microbial contamination increases, buffer substances are degraded, and the pH increases, which adversely affects the printing process, for example, a worse start-up behavior, increased water supply and unstable ink-water equilibrium.

During the printing operation, the microbial contamination in the dampening agent cycle can be prevented but insufficiently by adding biocides; germs are only inhibited in growth rather than killed by the biocides. The selection and concentration of these products is limited due to the health hazard to persons working on the printing machines.

When the microbial contamination of the dampening agent cycle is excessive, the whole system must be purified and rinsed with strongly alkaline solutions (for example, in combination with hydrogen peroxide, hypochlorite or other highly effective bactericides) for 5 to 8 hours. Such purification cycles are typically required every one to two months.

The problem of microbial contamination in the dampening agent cycle is the greater, the greater the printing machines and thus the circulation systems of the pipelines are. The whole liquid volume circulating in the cycle at a high flow rate is often within a range of from 200 to 500 liters in newspaper printing machines, a volume which must be transported over large distances. Newspaper machines are particularly susceptible to microbial contamination since their pipelines have a larger diameter and therefore are never completely filled with dampening agent. In addition, there is an enhanced load of paper dust, which largely consists of cellulose.

Bacteria which live freely in solution are relatively easily attacked and controlled by biocides or antibiotics. Once biofilms have formed, these are substantially more resistant. In the biofilms, the bacteria adhere to one another or to the substrate while being surrounded by extracellular substances which mainly consist of polysaccharides and proteins. This mucous layer protects the bacteria from external influences very effectively. With the commercially available biocides, the biofilms in the dampening solution piping systems of the printing machines cannot be securely controlled. Once germ coatings have formed in the dampening system, they grow on very rapidly and can interfere severely with the printing operation. Detached biofilms and agglomerates can be flushed into the dampening units where they clog filters, nozzles and valves. In the dampening agent reprocessing system, an increased germ growth can be noticed from a rotten smell in the dampening agent sump. A mucous layer forms on the filter mats of the return line and at the walls of the dampening agent sump.

Therefore, it was the object of the present disclosure to develop a process and a device with which the microbial contamination of dampening agent cycles can be counteracted.

### SUMMARY OF THE DISCLOSURE

This object is achieved by a process comprising the step of electrochemically treating the dampening agent. According to the disclosure, this is effected by applying a voltage between at least two electrodes in the dampening agent cycle.

This process serves to reduce germ growth in the dampening agent cycle. Thus, renewed microbial contamination can be inhibited or prevented, and even if microbial contamination already exists, a reduction of the germ count can be achieved.

In one embodiment, direct voltage can be employed, so that said at least two electrodes form an anode and cathode. In a preferred embodiment of the disclosure, the sump of the dampening agent reprocessing plant, which typically consists of stainless steel, is used as the cathode.

Alternatively and preferably, the voltage may also be an alternating voltage. The reversion of polarity avoids the production of cleavage products by electrolysis as well as the formation of depositions on the electrode surfaces. A square-wave voltage is a particularly suitable form of alternating voltage. The frequency of the alternating voltage is preferably within a range of from 0.01 to 108 Hertz, more preferably within a range of from 0.01 to 20,000 Hz, most preferably from 0.05 to 10 or from 1 to 10 Hz.

In both cases, when direct voltage or alternating voltage is used, the voltage is preferably within a range of from 0.5 to 250 Volt, preferably within a range of from 2 to 50 Volt, more preferably within a range of from 5 to 20 Volt. Since very high voltages mean risks to the operating personnel, lower voltage values are preferred.

Said at least two electrodes can be provided at any suitable site of the dampening agent cycle, for example, in the dampening agent supply line, in the circulation system, e.g., integrated in the pipeline system, or in the dampening agent sump. The electrochemical treatment of the dampening agent through the electrodes may also be effected from an external device. Thus, part of the dampening agent is treated in this device in bypass and then recycled. In principle, it is also possible to employ more than two electrodes or two or more pairs of electrodes provided at different sites.

Suitable materials for the electrodes may be selected from metals, semiconductors, conductive ceramics, graphite or conductive plastic materials. Particularly preferred materials for the electrodes include titanium, titanium alloys or coated titanium. Coatings of platinum, iridium, indium, ruthenium oxide, indium oxide, iridium oxide or mixtures thereof are particularly preferred.

It is assumed that oxygen free radicals form during the treatment; they have a much stronger effect on biofilms than chlorine, for example.

The controlling of microbial contamination includes the prevention of growth and the killing of the germs.

A clarification of the dampening agent is also observed. The dampening agent, which becomes rather turbid and colored in the course of the operation, becomes increasingly clearer during the treatment.

The treatment may be effected continuously, but it may also be interrupted for days or hours. In particular, the operation may be controlled depending on the growth of germs.

The disclosure also relates to a device for performing the process which comprises a dampening agent system with at least two electrodes and a power supply. A dampening agent system comprises dampening units, circulation lines and a dampening agent supply system. This dampening agent supply system comprises a dampening agent reprocessing device, a cooling device, a metering unit and a dampening agent sump. The dampening agent reprocessing device comprises pump units for the circulation and the filtering means.

### BRIEF DESCRIPTION OF THE DRAWINGS

FIG. 1 schematically shows an embodiment of the electrochemical treatment device.

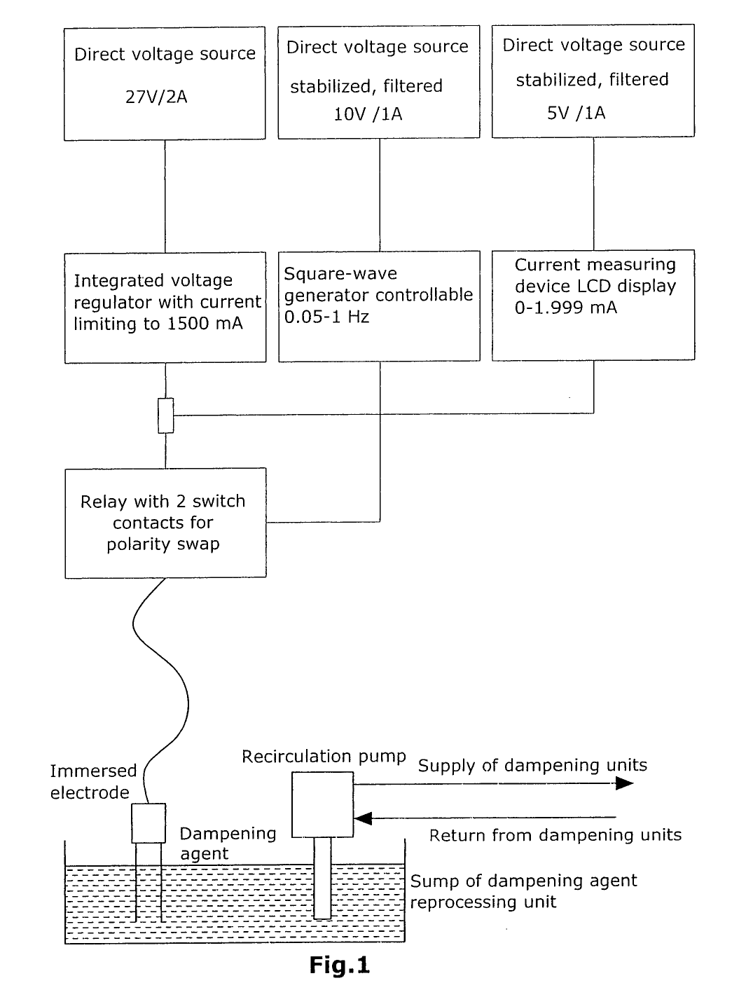

FIG. 2 shows an alternative embodiment of the electrochemical treatment device.

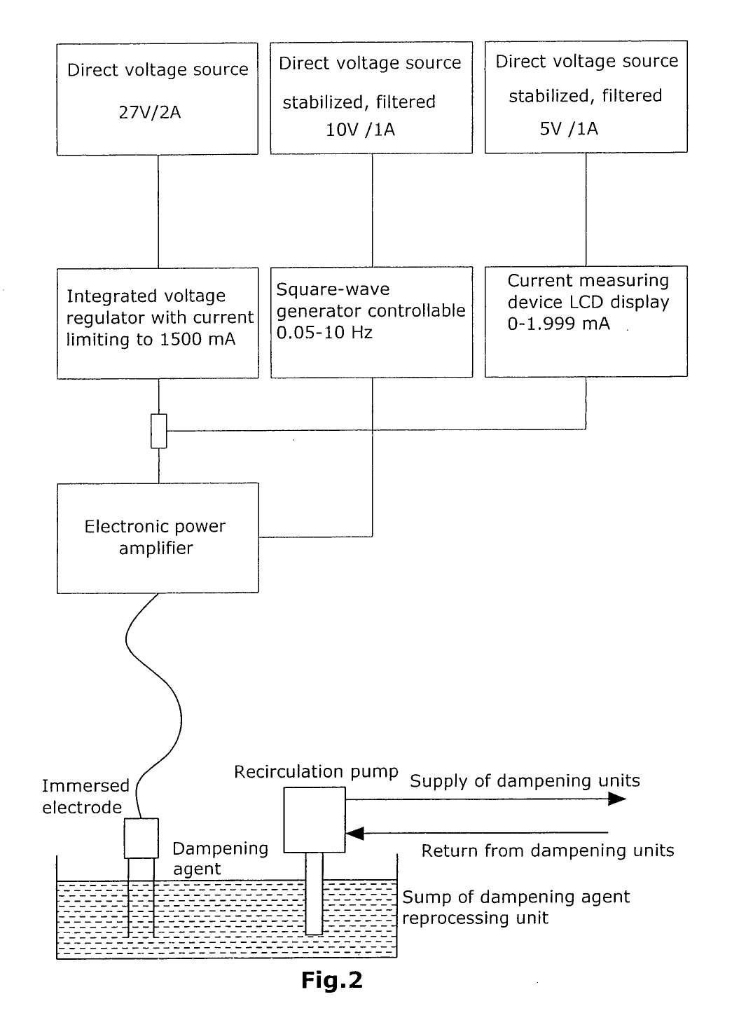

### DETAILED DESCRIPTION OF THE PREFERRED EMBODIMENT

The mode of operation of the system is further illustrated by the following Example. The set-up of the plant is as follows.

The required current for this plant is supplied through a transformer with a primary input voltage from the mains supply of from 230 to 240 V and alternating current outputs of 25 V, 12 V and 6 V. The transformer has a power of 100 W.

From the 25 V alternating current output, a direct voltage of 28 V is generated through a bridge rectifier and subsequent filtering with a 2200 pF electrolyte capacitor.

In order to protect the plant from overloads and short circuits, the output current is limited to 1.5 A by a controllable voltage regulator connected in series. In case of overload, the voltage regulator is heated and automatically downregulates the current.

From the 12 V alternating current output, a direct voltage of 14 V is generated through a bridge rectifier and stabilized to 10 V by an integrated voltage regulator. The filtering is effected by a 1000 pF electrolyte capacitor.

The stabilized 10 V direct voltage supplies the square-wave generator.

The components in the square-wave generator are dimensioned to obtain a pause-to-pulse ratio of 1:1. The square-wave generator can be regulated within a range of from 0.05 to 10 Hz through a potentiometer.

A relay is connected with the output of the square-wave generator. Through two switch contacts, the polarity of the 28 V direct voltage (item 2) is continuously swapped depending on the frequency of the square-wave generator to produce a square-wave alternating voltage.

The relay output is connected with the immersed electrode. The latter consists of two titanium sheets with dimensions of 300×30 mm and a thickness of 3 mm. The electric leads are cast in epoxy resin for protection. The electrode plates have a mutual distance of 10 mm.

From the 6 V alternating current output, a direct voltage of 8 V is generated through a bridge rectifier and stabilized to 5 V by an integrated voltage regulator. The filtering is effected by a 1000 pF electrolyte capacitor.

The stabilized 5 V direct voltage supplies the digital display for current measurement.

The current is measured by the voltage drop over a 1 Ohm resistor connected in series between the current limiter and the relay. The measuring range is from 0 to 1.999 mA.

In a test experiment, it was established that upon switching on the device, the current rises highly at first and then decreases after 1 to 2 hours and becomes stabilized. The current is highly dependent on the conductivity of the dampening agent. Preferably, the current is on the order of from 0.1 to 2 A, preferably from 0.1 to 0.6 A.

After two to four days, biofilms and other soil aggregates were increasingly detached from the pipelines and the circulation system, were flushed out and accumulated in the return solution on the filter mat. Further, it could be observed how the dampening solution became clearer from day to day.

The germ load was examined with so-called dip slides. The dip slides employed were Envirocheck Contact Slides of Merck, Darmstadt, Germany. The latter are agar-coated plates (casein peptone soymeal peptone agar). The backside of the dip slides contains the same coating with an added neutralizer which neutralizes the activity of the biocides in the dampening agent. The dip slides were incubated at 36° C. for three days and then evaluated.

The number of colonies formed (colony-forming units) is a measure of the germ load in the dampening agent (CFU/ml). Swab samples of both the filter and the dampening agent were examined. Further examinations were effected after 24 or 48 hours from the beginning of the electrochemical water treatment.

| | | without | with electrochemical | |
|---|---|---|---|---|
| | | electrochemical | water treatment | |
| Dip slides | | water treatment | after 24 h | after 48 h |
| Side 1, CFU/ml | filter | 10⁶-10⁷ | 10⁵ | 10³-10⁴ |
| | dampening | 10⁶ | 10³-10⁴ | 10² |
| | agent | | | |
| Side 2, cfu/ml, | filter | >10⁷ | 10⁵-10⁶ | 10⁴ |
| with neutralizer | dampening | 10⁶-10⁷ | 10⁴ | 10²-10³ |
| | agent | | | |

As can be seen, the germ load has decreased by several powers of ten after 24 hours of treatment according to the disclosure and thus reaches a value which is not critical in the dampening solution cycle. The process according to the disclosure did not affect the printing process and the properties of the dampening solution, such as pH value or conductivity.

The process according to the disclosure is suitable not only for dampening agents, but also for printing and purification cycles in flexographic printing.

It is particularly preferred to use the process according to the disclosure in water cycles in which there is a transfer of organic substances because the latter promote the growth of germs.

“Aqueous medium” means that the content of water is at least 50% by weight, preferably at least 70% by weight.

Another embodiment of the disclosure is a process for reducing microbial contamination of aqueous media in the printing field, comprising the step of electrochemically treating the aqueous medium, and a process for reducing microbial contamination in ultrafiltration systems and osmosis systems, comprising the step of electrochemically treating the aqueous medium.

For these processes, the same further embodiments are employed as described for the process for limiting microbial contamination in dampening agent cycles.

These further processes are suitable, in particular, for being performed in the printing field.

The printing field includes works in connection with the methods of letterpress printing, especially flexographic printing, gravure printing, planographic printing, especially offset printing, screen printing using printing machines by which printing inks and lacquers are applied to a printing substrate (paper, cardboard, paper-board, plastic, sheet metal, etc.) in an automated fashion.

1-27. (canceled)

28. A process for reducing the microbial contamination of cycles with aqueous media, comprising the step of electrochemically treating the aqueous medium, wherein said cycle is a dampening agent cycle.

29. The process according to claim 28, wherein said electrochemical treatment is effected by applying a voltage to at least two electrodes.

30. The process according to claim 28, wherein said electrochemical treatment is effected by applying a direct voltage, wherein said at least two electrodes form an anode and a cathode.

31. The process according to claim 29, wherein said voltage is an alternating voltage.

32. The process according to claim 31, wherein said alternating voltage is a square-wave, sinusoidal or triangular voltage.

33. The process according to claim 31, wherein the frequency of said alternating voltage is within a range of from 0.01 to 108 Hertz.

34. The process according to claim 29, wherein said voltage is within a range of from 0.5 to 250 Volt, preferably from 5 to 50 Volt, more preferably up to 25 Volt.

35. The process according to claim 29, wherein said at least two electrodes are provided in the dampening agent supply, in the circulation system or in a bypass.

36. The process according to claim 29, wherein the material for said at least two electrodes is independently selected from metals, semiconductors, conductive ceramics, graphite or conductive plastic materials.

37. The process according to claim 29, wherein the material for said at least two electrodes is independently selected from titanium, titanium alloys or coated titanium.

38. The process according to claim 37, wherein said coating of titanium is effected with platinum, iridium, indium, ruthenium oxide, indium oxide, iridium oxide or mixtures thereof.

39. A device for performing the process according to claim 1, comprising a circulation system, at least two electrodes and a power supply, wherein said circulation system is a dampening agent cycle and said power supply is an alternating voltage source.

40. A process for reducing the microbial contamination of aqueous media in the printing field, comprising the step of electrochemically treating the aqueous medium.

41. A process for reducing the microbial contamination in ultrafiltration systems and osmosis systems used in the printing field, comprising the step of electrochemically treating the aqueous medium.

42. The process according to claim 40, wherein said electrochemical treatment is effected by applying a voltage to at least two electrodes.

43. The process according to claim 41, wherein said electrochemical treatment is effected by applying a voltage to at least two electrodes.

44. The process according to claim 40, wherein said electrochemical treatment is effected by applying a direct voltage, wherein said at least two electrodes form an anode and a cathode.

45. The process according to claim 41, wherein said electrochemical treatment is effected by applying a direct voltage, wherein said at least two electrodes form an anode and a cathode.

46. The process according to claim 42, wherein said voltage is an alternating voltage.

47. The process according to claim 46, wherein said alternating voltage is a square-wave, sinusoidal or triangular voltage.

48. The process according to claim 46, wherein the frequency of said alternating voltage is within a range of from 0.01 to 108 Hertz.

49. The process according to claim 42, wherein said voltage is within a range of from 0.5 to 250 Volt, preferably from 5 to 50 Volt, more preferably up to 25 Volt.

50. The process according to claim 42, wherein the material for said at least two electrodes is independently selected from metals, semiconductors, conductive ceramics, graphite or conductive plastic materials.

51. The process according to claim 42, wherein the material for said at least two electrodes is independently selected from titanium, titanium alloys or coated titanium.

52. The process according to claim 51, wherein said coating of titanium is effected with platinum, iridium, indium, ruthenium oxide, indium oxide, iridium oxide or mixtures thereof.

53. The process according to claim 40, wherein said aqueous media in the printing field are water in the storage tanks of automated blanket and roller washing systems, water in the storage tanks of osmosis plants, and aqueous solutions in plate development systems.

54. The process according to claim 41, wherein said aqueous media in the printing field are water in the storage tanks of automated blanket and roller washing systems, water in the storage tanks of osmosis plants, and aqueous solutions in plate development systems.

55. The process according to claim 41, wherein said ultrafiltration systems and osmosis systems are ultrafiltration systems and/or osmosis systems used in the printing field.
